(19) United States
(12) Patent Application Publication
(10) Pub. No.: US 2020/0109368 A1
(43) Pub. Date: Apr. 9, 2020

(54) METHOD FOR PREPARING DIFFERENTIATION-INDUCED CELLS

(71) Applicants: TERUMO KABUSHIKI KAISHA, Tokyo (JP); OSAKA UNIVERSITY, Suita (JP)

(72) Inventors: Hiroko ISEOKA, Osaka (JP); Fumiya OHASHI, Kanagawa (JP); Yoshiki SAWA, Osaka (JP); Shigeru MIYAGAWA, Osaka (JP)

(73) Assignees: TERUMO KABUSHIKI KAISHA, Tokyo (JP); OSAKA UNIVERSITY, Suita (JP)

(21) Appl. No.: 16/703,036

(22) Filed: Dec. 4, 2019

Related U.S. Application Data
(63) Continuation of application No. PCT/JP2018/021441, filed on Jun. 5, 2018.

(30) Foreign Application Priority Data
Jun. 5, 2017 (JP) 2017-110952

(51) Int. Cl. C12N 5/077

(52) U.S. Cl. CPC C12N 5/0657; C12N 2533/20; C12N 2501/734; C12N 2506/45

(57) ABSTRACT

A method for preparing differentiation-induced cells from embryoid bodies derived from pluripotent stem cells is provided. The method includes adding a protease to embryoid bodies. The protease disperses the embryoid bodies, and the protease has an enzyme activity in the range from 0.3 to 4.0 recombinant protease activity unit (rPU)/ml.

### CROSS-REFERENCES TO RELATED APPLICATIONS

This application is a continuation of International Application No. PCT/JP2018/021441 filed on Jun. 5, 2018, which claims priority to Japanese Application No. 2017-110952 filed on Jun. 5, 2017, the entire content of both of which is incorporated herein by reference.

### TECHNICAL FIELD

The present invention generally relates to a method for purifying differentiation-induced cells derived from pluripotent stem cells, differentiation-induced cells purified by using the method, a sheet-shaped cell culture containing the differentiation-induced cells, particularly cardiomyocytes, and a method for treating a disease in a patient with the sheet-shaped cell culture.

### BACKGROUND DISCUSSION

Adult cardiomyocytes are poorly capable of self-renewal, and are very difficult to repair if myocardial tissue is damaged. In recent years, in order to repair damaged myocardial tissue, an attempt has been made to transplant a graft containing cardiomyocytes prepared by a cell engineering method to an affected area (Japanese Patent Application No. 2007-528755, and Shimizu et al., Circ Res. 2002 Feb. 22; 90(3): e40-e48). Recently, cardiomyocytes derived from pluripotent stem cells such as embryonic stem cells (ES cells) and induced pluripotent stem cells (iPS cells) have been considered as a source of cardiomyocytes used for preparation of such grafts and the preparation of a sheet-shaped cell culture containing such pluripotent stem cell-derived cardiomyocytes and experiments for treating animals have been attempted (Matsuura et al., Biomaterials. 2011 October; 32(30): 7355-62 and Kawamura et al., Circulation. 2012 Sep. 11; 126(11 Suppl 1): S29-37). However, sheet-shaped cell cultures containing cardiomyocytes derived from pluripotent stem cells have only just begun to be developed, and there are still many uncertainties regarding their functional characteristics and factors affecting them.

When preparing the differentiation-induced cells from pluripotent stem cells, for example, in the case of preparing cardiomyocytes, firstly, while providing directional differentiation into a mesoderm from pluripotent stem cells to form embryoid bodies, such embryoid bodies are induced to be differentiated into cardiomyocytes, and then cardiomyocytes are recovered by dispersing them into single cells (for example, Matsuura et al., Biomaterials. 2011 October; 32(30): 7355-62). With such dispersion, cardiomyocytes are recovered and various devices for efficiently recovering have been available.

### SUMMARY

A method is disclosed for purifying differentiation-induced cells derived from pluripotent stem cells, differentiation-induced cells purified using the method, a sheet-shaped cell culture including the differentiation-induced cells, and a method for treating a disease with the sheet-shaped cell cultures.

If cells induced to be differentiated from pluripotent stem cells are used for transplantation, it is essential to sort the induced cells and remove undifferentiated cells. If undifferentiated cells remain in the cell group to be transplanted, there is a risk that the undifferentiated cells become tumorigenic. As a method for removing undifferentiated cells when recovering cardiomyocytes from embryoid bodies induced to be differentiated into cardiomyocytes from the pluripotent stem cells, for example, the method described in International Patent Application Publication No. 2016/072519 is known.

In addition, in the case of using cells differentiated and derived from pluripotent stem cells for transplantation, it is desirable to prepare cells without using other animal-derived components. Therefore, as a method for preparing differentiation-induced cells for clinical use, it has become common to use the pluripotent stem cells prepared by feeder-free culture without feeder cells instead of conventional feeder culture.

While studying a method for preparing cardiomyocytes from pluripotent stem cells for clinical use, the inventors dispersed embryoid bodies prepared from clinical feeder-free pluripotent stem cell lines and used them for adhesion culture, but in this case, the inventors faced new problems in that the cell adhesion efficiency was worse compared to those prepared from feeder cell lines. As a result of continued research to solve these problems, it has been discovered that when the embryoid bodies are dispersed using an enzyme solution having a stronger activity than that conventionally used for dispersing the cells, the cell adhesion efficiency in the subsequent adhesion culture is improved. The inventors continued their research and arrived at the following aspects and embodiments.

An aspect is directed to a method for preparing differentiation-induced cells from an embryoid body derived from a pluripotent stem cell. The method includes adding a protease to embryoid bodies which disperses the embryoid bodies. The protease has an enzyme activity in the range from 0.3 to 4.0 recombinant protease activity unit (rPU)/ml.

In one embodiment, the protease has an enzyme activity of at least 0.45 rPU/ml.

In another embodiment, the protease has an enzyme activity from 0.9 to 1.2 rPU/ml.

In another embodiment, the protease is xeno free.

In another embodiment, the protease is TrypLE (registered trademark) Select.

In another embodiment, the method further includes treating the embryoid body with collagenase.

In another embodiment, the pluripotent stem cells are iPS cells.

In another embodiment, the pluripotent stem cells are human cells.

In another embodiment, the pluripotent stem cells are feeder-free cell lines.

In another embodiment, the differentiation-induced cell is a cardiomyocyte.

In another embodiment, a cell population having a troponin positive rate of 50 to 90% is obtained.

In another embodiment, the dispersed embryoid bodies have a diameter of 10 μm or more.

In another embodiment, the method further includes adhering the dispersed embryoid bodies to a culture substrate.

Another aspect is directed to a method of improving adhesion of embryoid bodies to a culture substrate. The method includes adding a protease to embryoid bodies. The protease disperses the embryoid bodies, and the protease has an enzyme activity in the range from 0.3 to 4.0 recombinant protease activity unit (rPU)/ml. The method further includes adhering the dispersed embryoid bodies to a culture substrate.

Another aspect is directed to a cardiomyocyte is prepared by a process that includes adding a protease to embryoid bodies derived from pluripotent stem cells. The protease disperses the embryoid bodies, the protease has an enzyme activity in the range from 0.3 to 4.0 recombinant protease activity unit (rPU)/ml, and the embryoid bodies are induced to be differentiated into cardiomyocytes. The method further includes adhering the dispersed embryoid bodies to a culture substrate.

According to another aspect, a clinical cell population induced to be differentiated from pluripotent stem cells can be obtained with higher efficiency and higher viability, thus providing an advantage over the prior art. In particular, when the embryoid bodies are dispersed, since the cells can then be recovered with high efficiency upon adhesion culture thereafter, and after embryoid bodies are formed, various purification methods for differentiation-induced cells using adhesion culture can be used.

### BRIEF DESCRIPTION OF DRAWINGS

FIG. 1 is a graph showing TnT positivity, viability, and cell number of a recovered cell population immediately after dispersal of an embryoid body when the embryoid body is treated with 1×triple select and treated with collagenase+Accumax. The left axis represents the proportion, the right axis represents the cell number, TnT positive represents the TnT positive rate, viability represents viability, and recovered cells represent the number of recovered cells.

FIG. 2 is a graph showing the TnT positive rate and the cell number of a recovered cell population immediately after an embryoid body dispersion when embryoid bodies are treated with 1×triple select, 3×triple select, and 10×triple select, respectively. The left axis represents the TnT positive rate, the right axis represents the number of recovered cells, TnT positive represents the TnT positive rate, and the recovered cells represent the number of recovered cells.

FIG. 3 is a graph showing the TnT positive rate, the change rate of TnT positive rate, and the cell recovery rate after adhesion culture of a recovered cell population obtained by dispersing embryoid bodies for 5 days when embryoid bodies are treated with 1×triple select and when treated with collagenase+Accumax. The left axis represents the TnT positive rate, the right axis represents the cell recovery rate, TnT positive represents the TnT positive rate, ▪ (closed square) represents the change rate of the TnT positive rate, and ▴ (closed triangle) represents the number of recovered cells.

FIGS. 4A, 4B and 4C are graphs showing the cell recovery rate (A), the change rate (B) of TnT positive rate, and the viability (C), respectively, after the recovered cell population obtained by dispersing embryoid bodies is applied to adhesion culture for 5 days, when the embryoid bodies are treated with 1×triple select, 3×triple select, and 10×triple select, respectively.

FIGS. 5A and 5B illustrate (A) a graph showing the viability, the cardiomyocytes purity, and recovered cells of the recovered cell population immediately after embryoid body dispersion, and (B) a graph showing the cell viability, cardiomyocytes purity, and a cell recovery rate after 5 days of adhesion culture of the recovered cell population, when the embryoid bodies are treated with 3×Triple select, collagenase+3×Triple select, and collagenase+10×Triple select. The left axis of the graph of A shows the proportion and the right axis shows the number of recovered cells. Viability represents the viability and recovery rate represents the recovery rate.

### DETAILED DESCRIPTION

Set forth below with reference to the accompanying drawings is a detailed description of embodiments of a method for purifying differentiation-induced cells derived from pluripotent stem cells, differentiation-induced cells purified by using the method, a sheet-shaped cell culture containing the differentiation-induced cells, and a method for treating a disease in a patient with the sheet-shaped cell culture stent, representing examples of the inventive methods, differentiation-induced cells and a sheet-shaped cell culture containing the differentiation-induced cells.

Unless defined otherwise herein, all technical and scientific terms have the same meaning as commonly understood by those skilled in the art. All patents, patent applications, and other publications and information referenced herein are hereby incorporated by reference in their entirety. In addition, in the case of conflict between the publication referred to in the present specification and the description in the present specification, the description in the present specification shall prevail.

In the present disclosure, “pluripotent stem cells” is a term well known in the art and means a cell having an ability to be differentiated into cells of all lineages belonging to three germ layers, i.e., endoderm, mesoderm, and ectoderm. Non-limiting examples of pluripotent stem cells include, for example, embryonic stem cells (ES cells), nuclear transplanted embryonic stem cells (ntES cells), induced pluripotent stem cells (iPS cells), and the like. Usually, when pluripotent stem cells are induced to be differentiated into specific cells, first, pluripotent stem cells are suspended and cultured to form aggregates of any of the above three germ layers and then to induce differentiation of cells forming aggregates into specific desired cells. In an embodiment, “embryoid body” means an aggregate of such cells.

In the present disclosure, “differentiation-induced cell derived from a pluripotent stem cell” means any cell that has been subjected to differentiation-inducing treatment so as to be differentiated from pluripotent stem cells into specific types of cells. Non-limiting examples of differentiation-induced cells include muscle cells such as cardiomyocytes and skeletal myoblasts, neural cells such as neuronal cells, oligodendrocytes, and dopamine producing cells, retinal cells such as retinal pigment epithelial cells, blood cells, cells of hematopoietic lineage such as bone marrow cells, immune related cells such as T cells, NK cells, NKT cells, dendritic cells, and B cells, cells constituting organs such as liver cells, pancreatic β cells, and kidney cells, chondrocytes, germ cells, and the like, as well as including precursor cells and somatic stem cells that differentiate into these cells. Examples of such precursor cells and somatic stem cells include, for example, mesenchymal stem cells in cardiomyocytes, multipotent cardiac progenitor cells, unipotent cardiac progenitor cells, neural stem cells in cells of nervous system, cells of hematopoietic system and immunity hematopoietic stem cells and lymphoid stem cells. Differentiation induction of pluripotent stem cells may be performed using any known method. For example, differentiation of pluripotent stem cells to cardiomyocytes may be induced based on the method described in Miki et al., Cell Stem Cell 16, 699-711, Jun. 4, 2015 or International Patent Application Publication No. 2014/185358.

The differentiation-induced cells may also be cells derived from iPS cells into which any useful gene other than a gene for reprogramming has been introduced. A non-limiting example of such cells includes, for example, T cells derived from iPS cells into which the gene of the chimeric antigen receptor described in Themeli M. et al. Nature Biotechnology, vol. 31, no. 10, pp. 928-933, 2013 has been introduced. In addition, cells into which any useful gene has been introduced after being induced to differentiate from pluripotent stem cells are also encompassed in the differentiation-induced cells of the present disclosure.

One aspect of the present disclosure relates to a method for preparing differentiation-induced cell from an embryoid body derived from pluripotent stem cells, the method includes dispersing the embryoid body with the use of a protease having an enzyme activity corresponding to 0.3 to 4.0 recombinant protease activity unit (rPU)/ml.

In the present disclosure, “preparation of differentiation-induced cells from embryoid bodies” refers to obtaining a cell population containing desired differentiation-induced cells from embryoid bodies. Also, “dispersing embryoid bodies” means making embryoid bodies (aggregates) become smaller structures. Examples of such structures include, for example, single cells, cell clusters, and the like, and preferably single cells. The size of the structures may be any size smaller than the original embryoid body, for example, a diameter of 100 μm or less, a diameter of 90 μm or less, a diameter of 80 μm or less, a diameter of 70 μm or less, a diameter of 60 μm or less, a diameter of 50 μm, a diameter of 40 μm or less, a diameter of 30 μm or less, a diameter of 20 μm or less, or a diameter of 10 μm or less.

In the present disclosure, “recombinant protease activity unit” or “rPU” is a unit that represents an amount of enzyme known in the art, and is disclosed in, for example, Nestler et al., Quest 2004; 1: 42-7, etc. 1 rPU is defined as the amount of enzyme capable of converting 1.0 mmol of acetylarginine paranitroaniline (Ac-Arg-pNA) substrate per minute at pH 8.0 and room temperature (22±1° C.).

Other known units showing the amount of protease enzyme are a TAME unit, a BAEE unit, a USP trypsin unit, and the like. One TAME unit is defined as the amount of enzyme capable of hydrolyzing 1 μmol of p-toluenesulfonyl-L-arginine methyl ester (TAME) per minute in the presence of 0.001 M calcium ion at pH 8.2 and 25° C. One BAEE unit is defined as the amount of enzyme that increases the absorbance at 253 nm (1 cm optical path length) per minute by 0.001 when Nα-benzoyl-L-arginine ethyl ester (BAEE) is used as a substrate at pH 7.6 and 25° C. One USP tryptic unit is defined as the amount of enzyme that increases the absorbance at 253 nm per minute by 0.003 when Nα-benzoyl-L-arginine ethyl ester (BAEE) is used as a substrate at pH 7.6 and 25° C.

Alternative units for these enzymes can be used by those skilled in the art. For example, 1 USP trypsin unit corresponds to 3 BAEE units. One TAME unit corresponds to about 57.5 BAEE units or about 19.2 USP trypsin units. One rPU corresponds to about 293 USP trypsin units.

The protease used in the method of the present disclosure has an enzyme activity corresponding to 0.3 to 4 recombinant protease activity units (rPU)/ml. By dispersing embryoid bodies using a protease having an enzyme activity in this range, the adhesion efficiency of the dispersed cells to the culture substrate can be enhanced, and as a result, differentiation-induced cells may be recovered with high efficiency.

The reason that adhesion efficiency of cells, dispersed with a protease having an enzyme activity in the range of the present invention, to a culture substrate is high, is not clear, but such a range may be considered to be the optimum range for embryoid body dispersion. That is, when the protease activity is lower than the lower limit value, cells forming the embryoid body may not be dispersed sufficiently, and conversely, when the protease activity is higher than the upper limit value, the dispersed cells themselves may be excessively damaged, and thus it is considered that, within the scope of the present invention, cells may be sufficiently dispersed without causing excessive damage to the cells.

The lower limit of the range of the protease activity of the present disclosure is not particularly limited as long as the embryoid body may be dispersed to a single cell. Non-limiting examples include 0.3 rPU/ml or more, 0.35 rPU/ml or more, 0.4 rPU/ml or more, 0.45 rPU/ml or more, 0.5 rPU/ml or more, 0.55 rPU/ml or more, 0.6 rPU/ml or more, 0.65 rPU/ml or more, 0.7 rPU/ml or more, 0.75 rPU/ml or more, 0.8 rPU/ml or more, 0.85 rPU/ml or more, 0.9 rPU/ml or more, 0.95 rPU/ml or more, 1.0 rPU/ml or more, and the like.

The upper limit of the range of protease activity of the present disclosure is not particularly limited as long as cells are not excessively damaged during dispersion. Non-limiting examples include 4.0 rPU/ml or less, 3.5 rPU/ml or less, 3.0 rPU/ml or less, 2.5 rPU/ml or less, 2.0 rPU/ml or less, 1.9 rPU/ml or less, 1.8 rPU/ml or less, 1.7 rPU or less, 1.6 rPU/ml or less, 1.5 rPU/ml or less, 1.4 rPU/ml or less, 1.3 rPU/ml or less, and 1.2 rPU/ml or less, and the like.

Therefore, a non-limiting example of the numerical value range of the protease activity of the present disclosure includes any combination of the upper limit value and the lower limit value exemplified above. That is, for example, 0.3 to 4.0 rPU/ml, 0.3 to 3.5 rPU/ml, 0.3 to 3.0 rPU/ml, 0.3 to 2.5 rPU/ml, 0.3 to 2.0 rPU/ml, 0.3 to 1.9 rPU/ml, 0.3 to 1.8 rPU/ml, 0.3 to 1.7 rPU/ml, 0.3 to 1.6 rPU/ml, 0.3 to 1.5 rPU/ml, 0.3 to 1.4 rPU/ml, 0.3 to 1.3 rPU/ml, 0.3 to 1.2 rPU/ml, 0.45 to 4.0 rPU/ml, 0.45 to 3.5 rPU/ml, 0.45 to 3.0 rPU/ml, 0.45 to 2.5 rPU/ml, 0.45 to 2.0 rPU/ml, 0.45 to 1.9 rPU/ml, 0.45 to 1.8 rPU/ml, 0.45 to 1.7 rPU/ml, 0.45 to 1.6 rPU/ml, 0.45 to 1.5 rPU/ml, 0.45 to 1.4 rPU/ml, 0.45 to 1.3 rPU/ml, 0.45 to 1.2 rPU/ml, 0.6 to 4.0 rPU/ml, 0.6 to 3.5 rPU/ml, 0.6 to 3.0 rPU/ml, 0.6 to 2.5 rPU/ml, 0.6 to 2.0 rPU/ml, 0.6 to 1.9 rPU/ml, 0.6 to 1.8 rPU/ml, 0.6 to 1.7 rPU/ml, 0.6 to 1.6 rPU/ml, 0.6 to 1.5 rPU/ml, 0.6 to 1.4 rPU/ml, 0.6 to 1.3 rPU/ml, 0.6 to 1.2 rPU/ml, 0.9 to 4.0 rPU/ml, 0.9 to 3.5 rPU/ml, 0.9 to 3.0 rPU/ml, 0.9 to 2.5 rPU/ml, 0.9 to 2.0 rPU/ml, 0.9 to 1.9 rPU/ml, 0.9 to 1.8 rPU/ml, 0.9 to 1.7 rPU/ml, 0.9 to 1.6 rPU/ml, 0.9 to 1.5 rPU/ml, 0.9 to 1.4 rPU/ml, 0.9 to 1.3 rPU/ml, 0.9 to 1.2 rPU/ml, and the like are included. Preferable is 0.6 to 2.0 rPU/ml and more preferable is 0.9 to 1.2 rPU/ml.

One of ordinary skill in the art may easily calculate how much given protease activity corresponds to rPU/ml, by using any relevant method and a conversion method known in the art. For example, it may be calculated by comparing the measured values (reference values) of liquid of a reference enzyme with known protease activity, with the conversion to the other units described above, for example, with the setting and measurement of another index, for example, the number of cells that may be seeded at confluence and may be detached in a predetermined time.

The protease that can be used in the method of the present disclosure may be any protease that can separate adherent cells, that is, a protease that can break intercellular adhesion. Non-limiting examples of proteases of the present disclosure include trypsin, chymotrypsin, thrombin, serine proteases such as elastase, collagenase, extracellular matrix degrading enzymes such as matrix metalloproteases, dispase, papain, pronase, and enzymes having the same activity, and in particular, enzymes from non-mammalian origin such as bacteria etc. These enzymes may be used alone or in combination of two or more.

Moreover, a commercially available product may be used as an enzyme for dispersion of a cell aggregate. Non-limiting examples of such products include, for example, TrypLE (registered trademark) Select and TrypLE (registered trademark) Express (both produced by ThermoFisher Scientific), dispase I and II (OENON Holdings, Inc. and Roche), Liberase (Roche), etc. In a preferred embodiment of the present disclosure, TrypLE (registered trademark) Select is used as a protease. TrypLE (registered trademark) Select is a recombinant enzyme obtained by microbial fermentation which contains no animal-derived component, and is marketed by ThermoFisher Scientific as a substitute for trypsin.

The inventors have found that when TrypLE (registered trademark) Select is used as a protease for dispersing embryoid bodies derived from pluripotent stem cells, the adhesion efficiency of the dispersed cells to the culture substrate is enhanced. Thus, in a preferred embodiment, the protease is TrypLE (registered trademark) Select. In another preferred embodiment, the step of dispersing embryoid bodies using a protease is followed by the step of dispersing embryoid bodies derived from pluripotent stem cells using collagenase. In yet another preferred embodiment, the step of dispersing the embryoid body derived from pluripotent stem cells using collagenase is followed by the step of dispersing the embryoid body using protease. By using collagenase in combination with the dispersion treatment with protease, the cell recovery rate immediately after dispersion and the purity of the target cell are increased, as compared with the case where the protease alone is dispersed, and the purity of the target cell may be further enhanced by the subsequent adhesion culture. In such an embodiment, the protease is preferably a protease other than collagenase.

In one embodiment of the present disclosure, pluripotent stem cells are, for example, embryonic stem cells (ES cells), nuclear transplanted embryonic stem cells (ntES cells), induced pluripotent stem cells (iPS cells), and the like. Preferably, the pluripotent stem cells are iPS cells.

In one embodiment of the present disclosure, pluripotent stem cells may be derived from any organism. Such organisms include, but are not limited to, humans, non-human primates, dogs, cats, pigs, horses, goats, sheep, rodents (e.g., mice, rats, hamsters, guinea pigs, etc.), rabbits, etc. Preferably, the pluripotent stem cells are human cells.

In one embodiment of the present disclosure, the target cell is a cell for application to a subject in need thereof. In the case where the subject is a specific animal, a series of steps in the method of producing a cell culture of the present disclosure is performed in an environment free of heterologous components. In the case where the subject is a human, a series of steps in the method of producing a cell culture of the present disclosure is performed in an environment free of non-human derived components. Thus, preferably, the proteases of the present disclosure are xeno free. In addition, as pluripotent stem cells of the present disclosure, feeder-free cell lines are preferably used.

The method of the present disclosure may be particularly suitably used in preparing cells to be employed in regenerative medicine using pluripotent stem cells. Thus, in one particularly preferred embodiment, the pluripotent stem cells are feeder-free cell lines of human iPS cells, and all the steps are carried out in a xeno free environment.

The method of the present disclosure may be used in the preparation of any differentiation-induced cells, including dispersing the embryoid bodies derived from pluripotent stem cells, and particularly in the preparation including performing adhesion culture after dispersion. Non-limiting examples of differentiation-induced cells that may be prepared by the method of the present disclosure include the cells listed in the above “differentiation-induced cells derived from pluripotent stem cell” and the like, with particular preference given to cardiomyocytes. The method for preparing the differentiation-induced cells of the present disclosure will be described in more detail below, using the case where the differentiation-induced cells are cardiomyocytes as an example, but the present disclosure should not be construed as being limited to such an embodiment.

The “cardiomyocytes derived from pluripotent stem cell” means cells having cardiomyocyte characteristics among differentiation-induced cells derived from pluripotent stem cell. Characteristics of cardiomyocytes include, but are not limited to, for example, the expression of cardiomyocyte markers, the presence of an autonomous beat, and the like. Non-limiting examples of cardiomyocyte markers include, for example, c-TNT (cardiac troponin T), CD172a (also known as SIRPA or SHPS-1), KDR (also known as CD309, FLK1, or VEGFR2), PDGFRA, EMILIN2, VCAM, etc. In one embodiment, cardiomyocytes derived from pluripotent stem cell are c-TNT positive and/or CD172a positive.

Various techniques are known for inducing cardiomyocytes from pluripotent stem cells (for example, Burridge et al., Cell Stem Cell. 2012 Jan. 6; 10 (1): 16-28) and also in any method, a mesodermal inducer (for example, activin A, BMP4, bFGF, VEGF, SCF, etc.), a cardiac specification factor (for example, VEGF, DKK1, a Wnt signal inhibitor (for example, IWR-1, IWP-2, IWP-3, IWP-4, etc.), BMP signal inhibitors (e.g., NOGGIN, etc.), TGFβ/activin/NODAL signal inhibitors (for example, SB431542, etc.), retinoic acid signal inhibitors) and cardiac differentiation factors (for example, VEGF, bFGF, DKK1, etc.) may enhance the induction efficiency by a sequential acting. In one embodiment, cardiomyocyte induction treatment from pluripotent stem cells is carried out by sequentially causing (1) a combination of BMP4, bFGF, and activin A, (2) VEGF and IWP-3, and (3) a combination of VEGF and bFGF to act on embryoid bodies formed by the action of BMP4.

A method of obtaining cardiomyocytes from human iPS cells is, for example, a method that includes the following steps:

(1) maintaining and culturing the established human iPS cells in a feeder cell-free medium (feeder free method);

(2) forming an embryoid body from the obtained iPS cells,

(3) culturing the obtained embryoid body in a culture solution containing activin A, bone morphogenetic protein (BMP) 4, and basic fibroblast growth factor (bFGF),

(4) culturing the obtained embryoid body in a culture solution containing a Wnt inhibitor, a BMP4 inhibitor, and a TGFβ inhibitor, and

(5) culturing the obtained embryoid body in a culture solution containing VEGF and bFGF.

In step (1), human iPS cells are maintained and cultured in a feeder cell-free medium, as described, for example, in International Patent Application Publication No. 2017/038562 and Nakagawa et al., Sci Rep. 2014; 4: 3594 (feeder free method). Specifically, methods include, for example, a method wherein Stem Fit AK03 (Ajinomoto Co., Inc.) is used as a culture medium, iPS cells are cultured and adapted on iMatrix 511 (Nippi, Incorporated), and maintenance culture is performed, and a method for performing passage as single cell wherein iPS cells are treated every 7 to 8 days with TrypLE (trademark) Select (Thermo Fisher Scientific K. K.).

After the above steps (1) to (5), a step (6) of optionally purifying the obtained cardiomyocytes may be selectively performed. The step of purifying cardiomyocytes includes, for example, a method of reducing non-target cells using a glucose free medium, a method of reducing undifferentiated cells using a heat treatment, and the like.

Using the foregoing method, an embryoid body derived from pluripotent stem cells including cardiomyocytes may be obtained. The obtained embryoid bodies may be further dispersed using a protease to obtain a cell population containing cardiomyocytes. Proteases that can be suitably used for such dispersion treatment are as previously described. The enzyme activity of the protease corresponds to 0.3 to 4.0 rPU/ml, preferably 0.6 to 3.2 rPU/ml, and more preferably 0.9 to 2.0 rPU/ml.

The cell population obtained by the method of the present embodiment contains a large number of cardiomyocytes, that is, troponin (c-TNT) positive cells. The troponin positive rate of the obtained cell population may be for example, 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, and 75% or more, and so on, but is not limited thereto.

Moreover, the troponin positive rate of the obtained cell population may be for example, 99% or less, 98% or less, 97% or less, 96% or less, 95% or less, 94% or less, 93% or less, 92% or less, 91% or less, 90% or less, 89% or less, 88% or less, 87% or less, 86% or less, 85% or less, 84% or less, 83% or less, 82% or less, 81% or less, 80% or less, and so on, but is not limited thereto.

Therefore, the range of the troponin positive rate of the cell population obtained may be any combination of the upper limit and the lower limit. In a preferred embodiment, the troponin positive rate of the cell population obtained is, for example, 50% to 90%, 55% to 90%, 60% to 90%, 65% to 90%, 70% to 90%, 75% to 90%, 50% to 85%, 55% to 85%, 60% to 85%, 65% to 85%, 70% to 85%, 75% to 85%, 50% to 80%, 55% to 80%, 60% to 80%, 65% to 80%, 70% to 80%, 75% to 80%, and so on.

The cardiomyocytes obtained by the method of the present embodiment are preferably used for cell transplantation as cardiomyocytes for regenerative medicine. Thus, pluripotent stem cells are preferably human cells, iPS cells, and/or feeder free cell lines. The protease used for the dispersion treatment is preferably a xeno-free protease which does not contain components derived from non-human animals.

A cell population obtained by dispersing embryoid bodies derived from pluripotent stem cells according to the method of the present embodiment may be further subjected to adhesion culture to purify desired differentiation-induced cells. The purification step may include increasing the desired content of differentiation-induced cells and removing cells having tumorigenicity. In the present disclosure, the “tumorigenic cells” means cells that can be transformed into tumor cells at the transplantation site after transplantation when transplanted into a subject. Non-limiting examples of “cells having tumorigenicity” include cells still having pluripotency (undifferentiated cells) even after differentiation-inducing treatment, and cells wherein genomic abnormalities occur, etc., and there are typically undifferentiated cells.

Removal of cells having tumorigenicity can be performed using methods known by one of ordinary skill in the art. Non-limiting examples of such techniques include various separation methods using markers specific to cells having tumorigenicity (e.g., cell surface markers, etc.) and drugs targeting the surface antigen of cells having tumorigenicity, and a heat treatment to reduce cells having tumorigenicity. In a preferred embodiment, the step of removing cells having tumorigenicity includes treating the surface antigen of cells having tumorigenicity with a targeted agent, and non-limiting examples, include the methods disclosed in International Patent Application Publication No. 2014/126146 and International Patent Application Publication No. 2012/056997, the method disclosed in International Patent Application Publication No. 2012/147992, the method disclosed in International Patent Application Publication No. 2012/133674, the method disclosed in International Patent Application Publication No. 2012/012803 (Japanese Application No. 2013-535194), the method disclosed in International Patent Application Publication No. 2012/078153 (Japanese Application No. 2014-501518), the methods disclosed in Japanese Application No. 2013-143968 and Tohyama S. et al., Cell Stem Cell Vol. 12 Jan. 2013, Page 127-137, the method disclosed in Lee MO et al., PNAS 2013 Aug. 27; 110 (35): E3281-90, the method disclosed in International Patent Application Publication No. 2016/072519, the method disclosed in International Patent Application Publication No. 2013/100080, the method disclosed in Japanese Application No. 2016-093178, the method using the heat treatment disclosed in International Patent Application Publication No. 2017/038526, and treatment with Brentuximab Vedotin disclosed in International Patent Application Publication No. 2016/072519. In a preferred embodiment of the present disclosure, removing cells having tumorigenicity includes a treatment with Brentuximab Vedotin.

Methods for increasing the content rate of desired differentiation-induced cells include various separation methods using markers specific to the desired differentiation-induced cells (e.g., cell surface markers, etc.), such as magnetic cell separation (MACS), a flow cytometry method, an affinity separation method, a method of expressing selection marker (e.g., antibiotic resistance gene, etc.) by specific promoter, a method utilizing auxotrophy of desired differentiation-induced cells, i.e., a method of destroying cells other than the desired differentiation-induced cells by culturing in a medium from which a nutrient source necessary for survival of cells other than the desired differentiation-induced cells is removed, a method of selecting cells capable of survival under poor nutrition conditions, and a method of recovering desired differentiation-inducing cells using difference in adhesion to a substrate coated with an adhesion protein other than cells and desired differentiation-induced cells, and further, combinations of these methods.

Methods of increasing the content rate of pluripotent stem cell-derived cardiomyocytes include various separation methods using markers specific to cardiomyocytes (for example, cell surface markers, etc.), such as magnetic cell separation (MACS), flow cytometry, affinity separation, a method of expressing a selection marker (e.g., antibiotic resistance gene, etc.) by a specific promoter, a method utilizing auxotrophy of cardiomyocytes, i.e., a method of destroying cells other than cardiomyocytes by culturing in a medium from which a necessary nutrient source necessary for survival of cells other than cardiomyocytes has been removed (Japanese Application No. 2013-143968), a method of selecting cells capable of survival under poor nutrition conditions (International Patent Application Publication No. 2007/088874), a method of selecting cardiomyocytes using a difference in affinity binding to adhesion proteins coated on the substrates between the cardiomyocytes and cells other than cardiomyocytes (Japanese Patent Application No. 2014-188180), and a combination of these methods (see, for example, such as those described in Burridge et al. above). Cell surface markers specific for cardiomyocytes include CD172a, KDR, PDGFRA, EMILIN2, VCAM etc. Moreover, promoters specific for cardiomyocytes include, for example, NKX2-5, MYH6, MLC2V, ISL1, etc. In one embodiment, cardiomyocytes are purified based on the cell surface marker CD172a.

As described above, the differentiation-induced cell obtained by the method of the present disclosure is any cell that is assumed to be applied to the target organ/part in need thereof. Thus, differentiation-inducing cells are cells applied to, as a non-limiting example, for example, heart, blood, blood vessels, a lung, a liver, a pancreas, a kidney, a large intestine, a small intestine, a spinal cord, a central nervous system, bone, eyes, skin, etc. In addition, the differentiation-induced cell of the present invention is applied to a subject for treating a disease. Accordingly, one aspect of the present disclosure relates to a cell culture or composition for treating a disease, including differentiation-induced cells prepared by the method of the present disclosure. Non limiting examples of diseases include heart diseases, blood diseases, blood vessel diseases, lung diseases, liver disease, pancreas disease, kidney diseases, large intestine diseases, small intestine diseases, spinal cord diseases, central nervous system disease, bone diseases, eye diseases, and skin diseases. When differentiation-induced cells are cardiomyocytes diseases include myocardial infarction (including chronic heart failure associated with myocardial infarction), dilated cardiomyopathy, ischemic cardiomyopathy, heart disease (e.g., heart failure, particularly chronic heart failure) with systolic dysfunction (e.g., left ventricular systolic dysfunction), and the like. For the disease, differentiation-induced cell and/or a sheet-shaped cell culture (cell sheet) of the differentiation-induced cell may be useful for treating the disease. Thus, in one embodiment of the present disclosure, a cell culture for treating a disease is a sheet-shaped cell culture.

In addition, another aspect of the present disclosure relates to a method of producing a sheet-shaped cell culture, which includes sheeting a cell population containing differentiation-induced cells prepared by the method of the present disclosure. The cell culture containing the differentiation-induced cells prepared by the method of the present disclosure is optionally frozen and thawed, and then makes a sheet shape, for example, as described in International Patent Application Publication No. 2017/010544 and the like.

In one embodiment, the method of producing a sheet-shaped cell culture of the present disclosure includes the following steps:

(i) preparing a cell population including desired differentiation-induced cells,

(ii) seeding the cell population obtained in step (i) onto a culture substrate,

(iii) shaping a sheet of the cell population seeded in step (ii) in a cell culture solution to form a sheet-shaped cell culture, and

(iv) detaching the sheet-shaped cell culture formed in step (iii) from the culture substrate.

In a preferred embodiment, the cells seeded in step (ii) are seeded at a density reaching confluence. In this case, “density reaching confluence” means a density at which the seeded cells do not substantially proliferate, and one skilled in the art can calculate the density to reach confluence in each cell. A specific non-limiting example of the density reaching confluency includes, for example, “the density in which proportion of cells contacting each other on the culture substrate immediately after the cells sediment on the culture substrate after seeding on the culture substrate is, for example, 90% or more of the whole cells”.

In the present disclosure, “sheet-shaped cell culture” refers to cells which are linked to each other in a sheet. The cells may be linked to each other directly (including via cell components such as adhesion molecules) and/or via an intermediary substance. The intermediary substance is not particularly limited as long as it is a substance that can link cells at least physically (mechanically), and examples include extracellular matrix. The intermediary substance is preferably of cell origin, in particular from cells constituting the sheet-shaped cell culture. The cells are at least physically (mechanically) linked, but may be further functionally linked, for example, chemically or electrically. The sheet-shaped cell culture is composed of one cell layer (monolayer), but is composed of two or more cell layers (layered (multilayer) body, for example, two layers, three layers, four layers, five layers, six layers, etc.). In addition, the sheet-shaped cell culture may have a three-dimensional structure having a thickness which exceeds a thickness of one cell without showing a clear layer structure of cells. For example, in the vertical cross section of the sheet-shaped cell culture, the cells may be present in a non-uniform (for example, mosaic) arrangement without uniformly aligning in the horizontal direction.

The sheet-shaped cell cultures of the present disclosure preferably do not include a scaffold (support). Scaffolds may be used to attach cells on and/or within their surface and maintain the physical integrity of sheet-shaped cell cultures, such as membranes and the like made of polyvinylidene difluoride (PVDF) known in the art, but the sheet-shaped cell culture of the present disclosure can maintain its physical integrity even without such scaffolds. In addition, the sheet-shaped cell culture of the present disclosure preferably includes only the substance derived from cells constituting the sheet-shaped cell culture, and does not contain any other substance.

The cells may be heterologous cells or homologous cells. In this case, “heterologous cells” mean cells derived from an organism of a species different from that of the recipient when a sheet-shaped cell culture is used for transplantation. For example, when the recipient is a human, cells derived from monkeys or pigs mean heterologous cells. Also, “homologous cells” mean cells derived from an organism of the same species as the recipient. For example, when the recipient is human, human cells mean homologous cells. Homologous cells include autologously derived cells (also referred to as autologous cells or autologous family-derived cells), i.e., cells derived from a recipient and homologous non-autologous cells (also referred to as cells of the other family). Autologous cells are preferred in the present disclosure as transplantation does not result in a rejection. However, it is also possible to use heterologous cells or homologous non-autologous cells. When heterologous cells or homologous non-autologous cells are used, immunosuppressive treatment may be required to suppress rejection. In the present specification, cells other than autologous cells, i.e., heterologous cells and homologous non-autologous cells may be collectively referred to as non-autologous cells. In one aspect of the present disclosure, the cells are autologous cells or homologous cells. In one aspect of the present disclosure, the cells are autologous cells. In another aspect of the present disclosure, the cells are homologous cells.

In a preferred embodiment, differentiation-induced cells are prepared from pluripotent stem cells by the preparation method described above. Non-limiting examples of pluripotent stem cells include, for example, embryonic stem cells (ES cells), nuclear transplanted embryonic stem cells (ntES cells), induced pluripotent stem cells (iPS cells), and the like. Non-limiting examples of differentiation-induced cells include muscle cells such as cardiomyocytes and skeletal myoblasts, neural cells such as neuronal cells, oligodendrocytes, and dopamine producing cells, retinal cells such as retinal pigment epithelial cells, blood cells, cells of hematopoietic lineage such as bone marrow cells, immune related cells such as T cells, NK cells, NKT cells, dendritic cells, and B cells, cells constituting organs such as liver cells, pancreatic β cells, kidney cells, etc., chondrocytes, germ cells, etc., as well as including precursor cells that differentiate into these cells, somatic stem cells, and cells into which other useful genes have been introduced before or after induction of differentiation.

Furthermore, differentiation-induced cells include any one or mixtures of two or more of the cells discussed above, such as hepatocytes, sinusoidal endothelial cells, Kupffer cells, stellate cells, pit cells, biliary epithelial cells, vascular endothelial cells, vascular endothelial precursor cells, fibroblasts, bone marrow-derived cells, adipose-derived cells, mesenchymal stem cells, and the like. Those skilled in the art may appropriately select useful differentiation-induced cells according to the desired purpose.

For the purpose of regeneration of kidney tissue, preparation of an artificial kidney simulating kidney tissue, or obtaining cells for evaluating kidney function, as examples of cells obtained by inducing differentiation from pluripotent stem cells, any one or mixtures of two or more of kidney cells, granule cells, collecting duct epithelial cells, parietal epithelial cells, podocytes, mesangial cells, smooth muscle cells, tubular cells, interstitial cells, glomerular cells, vascular endothelial cells, vascular endothelial precursor cells, fibroblasts, bone marrow-derived cells, adipose-derived cells, and mesenchymal stem cells are included. For the purpose of regeneration of adrenal tissue, preparation of artificial adrenal gland simulating adrenal gland, or obtaining cells for evaluating adrenal function, as examples of cells obtained by inducing differentiation from pluripotent stem cells, any one or mixtures of two or more of adrenal medulla cells, a adrenal cortex cells, spherical layer cells, fasciculata cells, reticuloepithelial cells, vascular endothelial cells, vascular endothelial precursor cells, fibroblasts, bone marrow-derived cells, adipose-derived cells, and mesenchymal stem cells are included.

When it is intended to obtain cells to be evaluated for skin regeneration or skin function, as examples of cells obtained by inducing differentiation from pluripotent stem cells, any one or mixtures of two or more of epidermal keratinocytes, melanocytes, nape muscle cells, hair follicle cells, vascular endothelial cells, vascular endothelial precursor cells, fibroblasts, bone marrow-derived cells, adipose-derived cells, and mesenchymal stem cells are included.

For the purpose of obtaining cells to evaluate mucosal tissue regeneration or the function of mucosal tissue, as examples of cells obtained by inducing differentiation from pluripotent stem cells, any one or mixtures of two or more of buccal mucosa, gastric mucosa, intestinal mucosa, olfactory cells, epithelial, oral mucosa and uterine mucosa are included.

For the purpose of obtaining cells to evaluate regeneration of the nervous system or nerve function, as examples of cells obtained by inducing differentiation from pluripotent stem cells, any one or mixtures of two or more of midbrain dopamine neurons, cerebral neurons, and retinal cells, cerebellar cells, and hypothalamic endocrine cell are included.

When it is intended to obtain cells that constitute blood, as examples of cells obtained by inducing differentiation from pluripotent stem cells, any one or mixtures of two or more of T cells, B cells, neutrophils, eosinophils, basophils, monocytes, platelets, and red blood cells are included.

The culture substrate is not particularly limited as long as cells can form a cell culture thereon, and includes, for example, containers of various materials, solid or semisolid surfaces in the container, and the like. The container is preferably made of a structure/material which does not allow liquid such as culture fluid to permeate. As examples of such materials, polyethylene, polypropylene, Teflon (registered trademark), polyethylene terephthalate, polymethyl methacrylate, nylon 6,6, polyvinyl alcohol, cellulose, silicon, polystyrene, glass, polyacrylamide, polydimethyl acrylamide, metals (for example, iron, stainless steel, aluminum, copper, and brass), and the like can be mentioned but are not limited to these. Also preferably, the container has at least one flat surface. As examples of such containers, culture vessels including a bottom surface including a culture substrate capable of forming a cell culture, and a liquid impermeable side surface may be mentioned but are not limited to these. As specific examples of such culture containers, cell culture dishes, cell culture bottles, and the like may be mentioned without limitation. The bottom of the container may be transparent or opaque. When the bottom of the container is transparent, cells may be observed, counted, etc. from the bottom of the container. Also, the container may have a solid or semi-solid surface inside. Those having solid surfaces include plates and containers formed of various materials as described above, and semi-solid surfaces include gels, flexible polymer matrices, and the like. The culture substrate may be produced using the above-mentioned material, or a commercially available one may be used. Preferred culture substrates include, without limitation, for example, substrates having an adhesive surface suitable for forming sheet-shaped cell cultures. Specifically, a substrate having a hydrophilic surface include, for example, a substrate coated on the surface with a hydrophilic compound such as corona discharge treated polystyrene, collagen gel, or hydrophilic polymer, furthermore, substrates coated on the surfaces with extracellular matrices such as collagen, fibronectin, laminin, vitronectin, proteoglycan, and glycosaminoglycan, and a cell adhesion factor such as cadherin family, selectin family, integrin family, etc. Such substrates are also commercially available (e.g., Corning (registered trademark) TC-Treated Culture Dish, Corning, etc.). The culture substrate may be transparent or opaque in whole or in part.

The culture substrate may be coated on the surface with a material whose physical properties change in response to a stimulus such as temperature or light. Without particular limitation, for example, (meth) acrylamide compounds, N-alkyl substituted (meth) acrylamide derivatives (e.g., N-ethyl acrylamide, N-n-propyl acrylamide, N-n-propyl methacrylamide, N-isopropyl acrylamide, N-isopropyl methacrylamide, N-cyclopropyl acrylamide, N-cyclopropyl methacrylamide, N-ethoxyethyl acrylamide, N-ethoxyethyl methacrylamide, N-tetrahydrofurfuryl acrylamide, N-tetrahydrofurfuryl methacrylam ide, etc.), N,N-dialkyl substituted (meth) acrylamide derivatives (e.g., N, N-dimethyl (meth) acrylamide, N,N-ethylmethyl acrylamide, N,N-diethyl acrylamide, etc.), (meth) acrylamide derivatives having a cyclic group (for example, 1-(1-oxo-2-propenyl)-pyrrolidine, 1-(1-oxo-2-propenyl)-piperidine, 4-(1-oxo-2-propenyl)-morpholine, 1-(1-oxo-2-methyl-2-) propenyl)-pyrrolidine, 1-(1-oxo-2-methyl-2-propenyl)-piperidine, 4-(1-oxo-2-methyl-2-propenyl)-morpholine, etc.), or known materials such as a thermoresponsive material consisting of homopolymers or copolymers of vinyl ether derivatives (e.g., methyl vinyl ether), light absorbing polymers having azobenzene groups, copolymers of vinyl derivatives of triphenylmethane leuco hydroxide and acrylamide monomers, and a photoresponsive material such as an N-isopropylacrylamide gel containing spirobenzopyran may be used (see, for example, Japanese Application Publication No. 2-211865 and Japanese Application No. 2003-33177). By applying a predetermined stimulus to these materials, their physical properties, such as hydrophilicity and hydrophobicity, may be changed to promote detachment of the cell culture adhered on the same materials. Culture dishes coated with a thermoresponsive material are commercially available (e.g., UpCell (registered trademark) from CellSeed Inc.), and may be used in the manufacturing method of the present disclosure.

The culture substrate may have various shapes, but is preferably flat. Also, the area is not particularly limited, and may be, for example, about 1 cm² to about 200 cm², about 2 cm² to about 100 cm², about 3 cm² to about 50 cm², or the like.

The culture substrate may be coated (coating) with serum. Higher density sheet-shaped cell cultures can be formed by using culture substrates coated with serum. The expression “coated with serum” means that the serum component is attached to the surface of the culture substrate. Such conditions can be obtained without limitation, for example, by treating the culture substrate with serum. Treatment with serum involves contacting the serum with the culture substrate and, if necessary, incubating for a predetermined period of time.

As a serum, heterologous serum and/or homologous serum can be used. Heterologous serum means serum derived from an organism of a species different from that of the recipient when the sheet-shaped cell culture is used for transplantation. For example, when the recipient is a human, serum derived from cows and horses, such as fetal bovine serum (FBS, FCS), calf serum (CS), equine serum (HS), etc. means heterologous serum. Also, “homologous serum” means serum derived from an organism of the same species as the recipient. For example, when the recipient is human, human serum means homologous serum. Homologuous serum includes autologously derived serum (also referred to as autologous family-derived serum), i.e., serum derived from the recipient and homologous serum derived from the same species of individuals, other than the recipient. In the present specification, serum other than autologous serum, that is, heterologous serum, and homologous serum may be collectively referred to as non-autologous serum.

Serum for coating a culture substrate is commercially available or may be prepared by a routine method from blood collected from a desired organism. Specifically, for example, methods of coagulating collected blood at room temperature for about 20 minutes to about 60 minutes, centrifuging this at about 1000×g to about 1200×g and collecting the supernatant may be mentioned.

When incubated on a culture substrate, serum may be used as a stock solution or may be used after dilution. Dilutions may be carried out by any medium, including, but not limited to, water, physiological saline, various buffers (e.g., PBS, HBSS, etc.), various liquid media (e.g., DMEM, MEM, F12, DMEM/F12, DME, RPMI 1640, MCDB (MCDB 102, 104, 107, 120, 131, 153, 199, etc.), L15, SkBM, RITC 80-7, etc.). The dilution concentration is not particularly limited as long as the serum components can be deposited on the culture substrate, and for example, about 0.5% to about 100% (v/v), preferably about 1% to about 60% (v/v), and more preferably about 5% to about 40% (v/v).

The incubation time is also not particularly limited as long as the serum components can be deposited on the culture substrate, and for example, about 1 hour to about 72 hours, preferably about 4 hours to about 48 hours, more preferably about 5 hours to about 24 hours, and still more preferably about 6 hours to about 24 hours. The incubation temperature is also not particularly limited as long as the serum components can be deposited on the culture substrate, and for example, about 0° C. to about 60° C., preferably about 4° C. to about 45° C., and more preferably room temperature to about 40° C.

The serum may be discarded after incubation. As a serum disposal method, a conventional liquid disposal method such as aspiration with a pipette or decantation can be used. In a preferred embodiment of the present disclosure, the culture substrate may be washed with a serum-free washing solution after serum disposal. The serum-free washing solution is not particularly limited as long as it is a liquid medium that does not contain serum and does not adversely affect serum components attached to the culture substrate, and can be for example, without limitation, water, physiological saline, various buffers solution (e.g., PBS, HBSS, etc.), various liquid media (e.g., DMEM, MEM, F12, DMEM/F12, DME, RPMI 1640, MCDB (MCDB 102, 104, 107, 120, 131, 153, 199, etc.), L15, SkBM, RITC 80-7, etc.). For example, without limitation, a washing method is a conventional culture substrate washing method wherein a serum-free washing solution can be used by adding it to the culture substrate with stirring for a predetermined time (for example, about 5 seconds to about 60 seconds) and then discarding.

Another aspect of the present disclosure relates to a methods of treating a disease in a subject including applying an effective amount of a cell culture, a composition, or a sheet-shaped cell culture containing the differentiation-induced cells of the present disclosure to the subject in need thereof. The diseases to be treated are as previously described.

In the present disclosure, the term “treatment” is intended to encompass all types of medically acceptable prophylactic and/or therapeutic interventions aimed at curing, temporary remission, or prevention of disease. For example, the term “treatment” refers to medically acceptable interventions for a variety of purposes, and includes delaying or halting the progression of a disease associated with tissue abnormalities, providing regression or elimination of a lesion, preventing the onset of the disease, or preventing relapse.

In the treatment method of the present disclosure, a component that enhances the viability, engraftment and/or function of cell cultures, compositions, or sheet-shaped cell cultures, and other active ingredients useful for treating a target disease, etc. can be used in combination with the cell culture, composition, sheet-shaped cell culture, and the like of the present disclosure.

The treatment method of the present disclosure may further include the step of producing the sheet-shaped cell culture of the present disclosure according to the manufacturing method of the present disclosure. The treatment method of the present disclosure includes collecting cells for producing a sheet-shaped cell culture from a subject (for example, skin cells, blood cells, and the like when using iPS cells) or a tissue (e.g., skin tissue, blood, etc. when using iPS cells) from which cells are derived prior to the step of producing the sheet-shaped cell culture. In one embodiment, the subject from which the cells or tissue thereof are to be collected is the same individual as the subject to which the cell culture, the composition, the sheet-shaped cell culture, or the like is administered. In another embodiment, the subject from which the cells or tissue thereof are to be collected is a separate entity of the same as the subject to which the cell culture, the composition, the sheet-shaped cell culture, or the like is administered. In another embodiment, the subject from which the cells or tissue thereof is collected is an individual that is heterologous to the subject to which the cell culture, the composition, or the sheet-shaped cell culture is administered.

In the present disclosure, an effective amount is, for example, an amount capable of suppressing the onset or relapse of a disease, reducing symptoms, or delaying or stopping the progression (for example, size, weight, number of sheets of sheet-shaped cell culture, etc.), and preferably, an amount that prevents the onset and relapse of the disease or cures the disease. Also, it is preferably an amount that does not adversely affect the benefits of administration. Such amount may be determined as appropriate, for example, with testing in experimental animals such as mice, rats, dogs, or pigs, or in disease model animals, and such test methods are well known to those skilled in the art. In addition, the size of the tissue lesion to be treated can be an important indicator for determining the effective amount.

Methods for administration include, for example, intravenous administration, intramuscular administration, intraosseous administration, intrathecal administration, direct application to tissues, and the like. Although the frequency of administration is typically once per treatment, multiple administrations can also be performed if the desired effect cannot be obtained. When applied to a tissue, the cell culture, composition, sheet-shaped cell culture, or the like of the present disclosure may be fixed to the target tissue by a locking means such as sutures or staples.

### EXAMPLES

The invention will be described in more detail with reference to the following examples, which illustrate specific examples of the invention, and is not limited thereto.

In the following examples, as pluripotent stem cells, clinical human iPS cells established at Kyoto University iPS Cell Research Institute (CiRA) were used (see M. Nakagawa et al., Scientific Reports, 4:3594 (2014) and was maintained by the feeder-free method. In addition, embryoid bodies were obtained by inducing differentiation into cardiomyocytes with reference to the description of Miki et al., Cell Stem Cell 16, 699-711, Jun. 4, 2015, International Patent Application Publication No. 2014/185358, and International Patent Application Publication No. 2017/038562. Specifically, human iPS cells maintained and cultured in a feeder-free culture medium were cultured for 1 day in Stem Fit AK03 medium (Ajinomoto Co., Inc.) containing 10 μM Y27632 (Wako Pure Chemical Industries, Ltd.) on EZ Sphere (AGC Inc.) and the obtained embryoid body was cultured in a culture solution containing activin A, bone morphogenetic protein (BMP) 4, and basic fibroblast growth factor (bFGF), and further cultured in a culture solution containing a Wnt inhibitor (IWP3), a BMP4 inhibitor (Dorsomorphin), and a TGFβ inhibitor (SB431542), followed by culturing in a medium containing VEGF and bFGF.

### Example 1

### Evaluation of Dispersion of Embryoid Bodies Into Single Cells

The embryoid bodies containing cardiomyocytes after induction of differentiation were dispersed into single cells by adding the dispersion solution and incubating at 37° C. As a dispersion solution, a stock solution of TrypLE (trademark) Select Enzyme (10×), no phenol red (manufactured by Thermo Fisher Scientific K. K.) (hereinafter referred to as Triple Select or TS), or a solution obtained by diluting the stock solution to 30% concentration with 1 mM EDTA (3×TS) or a solution (1×TS) diluted to 10% concentration, or 2 mg/ml collagenase and Accumax (innovative cell technologies) were used. Incubation was carried out at 37° C. for 10 to 15 minutes in case of Triple Select, or incubation was carried out in collagenase for 1 hour at 37° C. in case of collagenase and Accmax, and thereafter collagenase was removed, Accumax was added, and incubation was performed for 10 to 15 minutes. The number of recovered cells and viability were calculated by performing trypan blue staining on dispersed single cells. For cardiomyocyte purity, dispersed cells are fixed and permeabilized using BD Cytofix/Cytoperm (trademark) Fixation/Permeabilization Solution Kit (manufactured by BD), and then anti-human troponin antibody (manufactured by Thermo Fisher Scientific K. K.), labeled secondary antibody (manufactured by Thermo Fisher Scientific K. K.) after sequentially reacting them (manufactured by Thermo Fisher Scientific K. K.), and were measured by a flow cytometer and calculated as a troponin (TnT) positive rate.

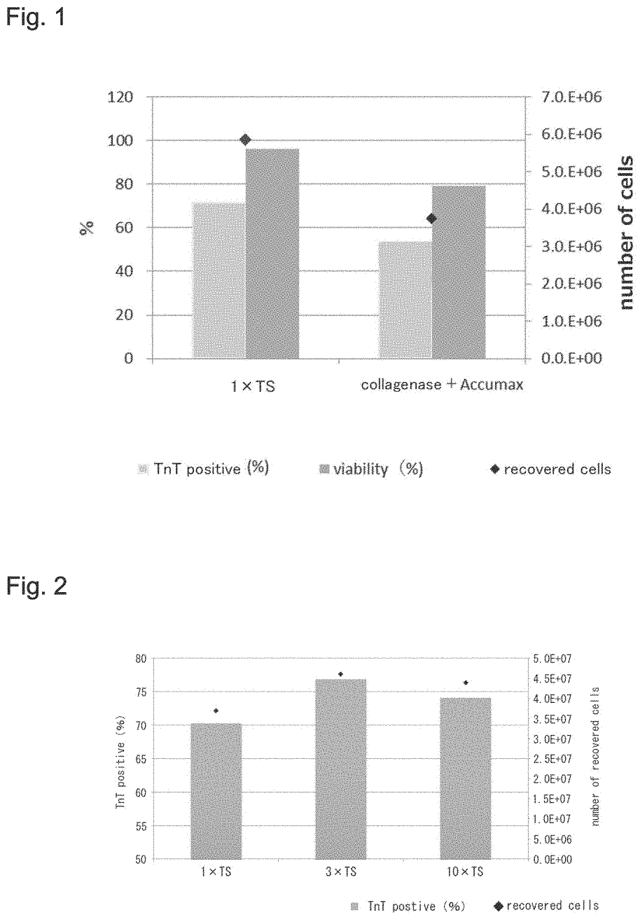

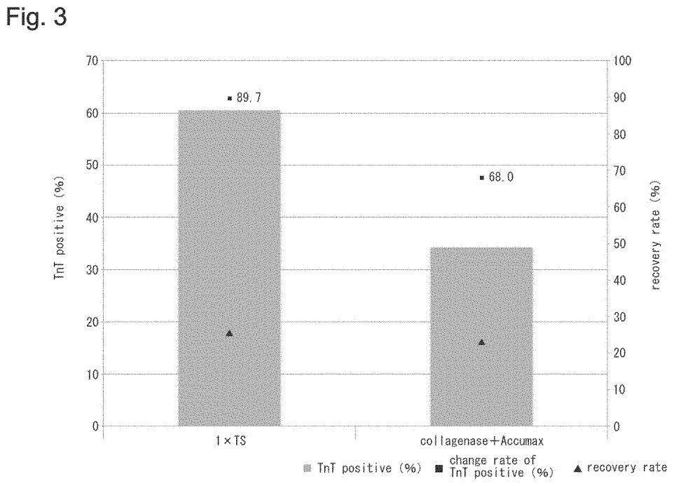

The results are shown in FIGS. 1 and 2. FIG. 1 is a graph comparing collagenase+Accumax and 1×TS. In comparison with collagenase+Accumax, 1×TS worked well in terms of the number of recovered cells, viability and cardiomyocyte purity. FIG. 2 is a graph comparing the results of 1×TS, 3×TS, and 10×TS. The number of recovered cells and cardiomyocyte purity were better in the case of using 3×TS and 10×TS as compared to 1×TS, and in terms of the number of recovered cells and cardiomyocyte purity was the best in the case of using 3×TS.

### Example 2

### Evaluation of Adhesion Culture After Dispersing Embryoid Bodies into Single Cells

The cells dispersed into single cells in Example 1 were seeded at a density of 1.8×10⁶ cells/cm² in culture dishes coated with 0.1% gelatin and cultured for 5 days. After 5 days, cells were recovered using 1×TS, and the cells were counted and viability was calculated by trypan blue staining. The recovery rate was calculated from the number of viable cells recovered relative to the number of seeded cells. After fixing the dispersed cells, the cardiomyocyte was sequentially reacted with an anti-human troponin antibody and a labeled secondary antibody in the same manner as above, and then the purity was measured by a flow cytometer to calculate a troponin (TnT) positive rate. The percentage change in cardiomyocyte purity (TnT positive rate) was calculated as cardiomyocyte purity after culturing for 5 days, assuming that the cardiomyocyte purity before seeding in a culture dish was 100%.

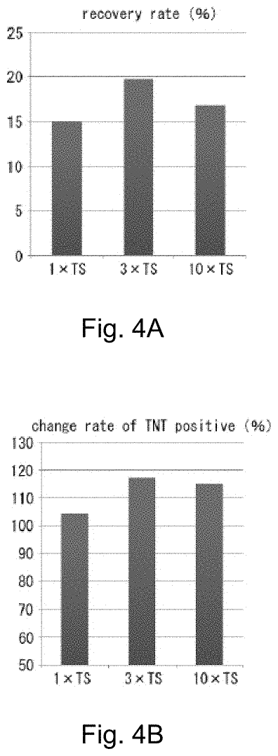

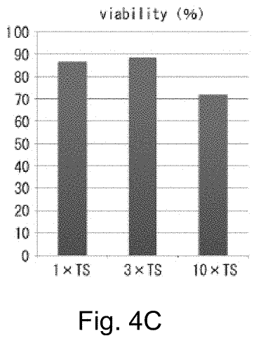

The results were shown in FIGS. 3 and 4. FIG. 3 is a graph comparing the case where cells dispersed with collagenase+Accumax were seeded and the case where cells dispersed with 1×TS were seeded. Cell recovery rates were almost the same in both cases, but 1×TS worked well in terms of cardiomyocyte purity and cardiomyocyte purity change rate. This indicates that when the cells in which the embryoid bodies were dispersed were further subjected to adhesion culture, the recovery amount of cardiomyocytes was significantly increased if the embryoid bodies were dispersed by Triple Select. Further, FIG. 4 is a graph comparing the results of dispersion at 1×TS, 3×TS, and 10×TS. Both cell recovery rate and cardiomyocyte purity change rate were good in 3×TS and 10×TS as compared to 1×TS, but cell viability was better in the case of using 1×TS, and 3×TS than the case of using 10×TS. In general, when 3×TS was used, the cell recovery rate, the change rate of cardiomyocyte purity, and the viability were all the best.

### Example 3

### Combined Use with Collagenase

In the dispersion of embryoid bodies, the difference in the effects of using Triple Select alone and using Triple Select and collagenase in combination was compared. Similarly to the combination of collagenase and Accumax in Example 1, the combination of Triple Select and collagenase was performed using Triple Select instead of Accumax. The comparison immediately after the dispersion was the same as in Example 1, and the comparison after the culture was the same as in Example 2.

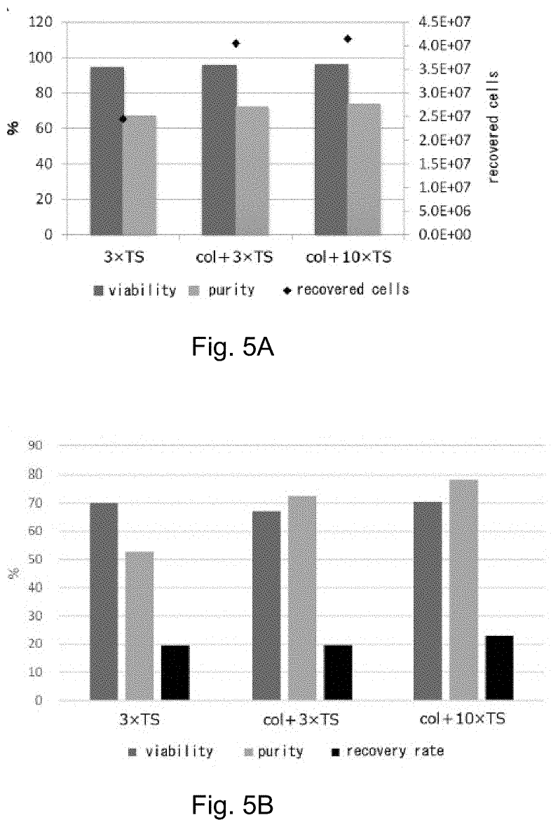

The results are shown in FIG. 5. Compared with triple select alone, combined use with collagenase showed no significant difference in viability immediately after dispersion, but the number of recovered cells tended to increase and the purity of cardiomyocytes tended to be high. Also, after contact culture, there was no significant difference in cell recovery rate and viability, but cardiomyocyte purity tended to increase.

### INDUSTRIAL APPLICABILITY

The methods, differentiation-induced cells and sheet-shaped cell culture containing differentiation-induced cells described above by way of example make it possible to efficiently prepare differentiation-induced cells from embryoid bodies in inducing differentiation of cells from pluripotent stem cells. In particular, in feeder-free cell lines, as compared to feeder cell lines, it is difficult to adhere to culture substrates in adhesion culture after dispersing embryoid bodies into single cells, resulting in low cell recovery after adhesion culture, and according to the method, when purifying a target differentiation-induced cell by adhesion culture, the target differentiation-induced cell may be obtained with higher efficiency than in the related art.

The detailed description above describes embodiments of methods, differentiation-induced cells and sheet-shaped cell culture containing differentiation-induced cells representing examples of the inventive methods, differentiation-induced cells and sheet-shaped cell culture containing differentiation-induced cells disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A method for preparing differentiation-induced cells from embryoid bodies derived from pluripotent stem cells, the method comprising:
adding a protease to embryoid bodies such that the protease disperses the embryoid bodies, the protease having an enzyme activity in a range from 0.3 to 4.0 recombinant protease activity unit (rPU)/ml.

2. The method according to claim 1, wherein the protease has an enzyme activity of at least 0.45 rPU/ml.

3. The method according to claim 1, wherein the protease has an enzyme activity from 0.9 to 1.2 rPU/ml.

4. The method according to claim 1, wherein the protease is xeno free.

5. The method according to claim 1, wherein the protease is TrypLE (registered trademark) Select.

6. The method according to claim 1, further comprising treating the embryoid body with collagenase.

7. The method according to claim 1, wherein the pluripotent stem cells are induced pluripotent stem (iPS) cells.

8. The method according to claim 1, wherein the pluripotent stem cells are human cells.

9. The method according to claim 1, wherein the pluripotent stem cells are feeder-free cell lines.

10. The method according to claim 1, wherein the differentiation-induced cell is a cardiomyocyte.

11. The method according to claim 1, wherein a cell population having a troponin positive rate of 50 to 90% is obtained.

12. The method according to claim 1, wherein the dispersed embryoid bodies have a diameter of 10 μm or more.

13. The method according to claim 1, further comprising adhering the dispersed embryoid bodies to a culture substrate.

14. A method of improving adhesion of embryoid bodies to a culture substrate comprising:
adding a protease to embryoid bodies such that the protease disperses the embryoid bodies, the protease having an enzyme activity in a range from 0.3 to 4.0 recombinant protease activity unit (rPU)/ml; and
adhering the dispersed embryoid bodies to a culture substrate.

15. The method according to claim 14 wherein the protease has an enzyme activity from 0.9 to 1.2 rPU/ml.

16. The method according to claim 14, wherein the protease is TrypLE (registered trademark) Select.

17. The method according to claim 14, further comprising treating the embryoid body with collagenase.

18. A cardiomyocyte prepared by a method comprising:
adding a protease to embryoid bodies derived from pluripotent stem cells to cause the protease to disperse the embryoid bodies, the protease having an enzyme activity in the range from 0.3 to 4.0 recombinant protease activity unit (rPU)/ml, and wherein the embryoid bodies are induced to be differentiated into cardiomyocytes; and
adhering the dispersed embryoid bodies to a culture substrate.

19. The cardiomyocyte according to claim 18, wherein the protease has an enzyme activity from 0.9 to 1.2 rPU/ml.

20. The cardiomyocyte according to claim 18, wherein the method further comprises treating the embryoid body with collagenase.
